(19) United States
(12) Patent Application Publication
(10) Pub. No.: US 2024/0290424 A1
(43) Pub. Date: Aug. 29, 2024

(54) Computational Characterization and Selection of Sequence Variants

(71) Applicant: EVQLV, INC., Surfside, FL (US)

(72) Inventors: Brett Anthony AVERSO, Surfside, FL (US); Andrew Charles SATZ, Surfside, FL (US)

(21) Appl. No.: 18/572,136

(22) PCT Filed: Jun. 21, 2022
(86) PCT No.: PCT/US2022/034256

§ 371 Date: Dec. 19, 2023

Related U.S. Application Data
(60) Provisional application No. 63/213,631, filed on Jun. 22, 2021.
(60) Provisional application No. 63/213,645, filed on Jun. 22, 2021.

(51) Int. Cl. G16B 20/20; G16B 15/30; G16B 40/20

(52) U.S. Cl. CPC G16B 20/20; G16B 15/30; G16B 40/20

(57) ABSTRACT

Data is received that characterizes a candidate pool of variants of sequences. Thereafter, sequence liabilities are calculated for each of the variants in the candidate pool. Variants from the candidate pool that possess more sequence liabilities than an antibody seed sequence are eliminated. Later, structural properties of all variants in the candidate pool are computationally characterizing using at least one machine learning model. Biophysical patches are then characterized using predicted structural properties for each of the variants in the candidate pool. A statistical divergence of a structure of each of the variants in the candidate pool is calculated relative to the structure of at least one sequence of interest. The variants in the candidate pool are computationally screened using the structural properties and biophysical patches of the variants to result in screened variants. Data can then be provided which characterizes the screened variants.

### TECHNICAL FIELD

The subject matter described herein relates to computationally characterization and selection of protein sequences for diagnostic and therapeutic drug discovery applications.

### BACKGROUND

Monoclonal antibodies are man-made proteins that act like antibodies of the immune system. Conventional laboratory methods for the identification of monoclonal antibodies within drug discovery involve hybridoma technologies or display technologies. The antibodies which comprise our immune repertoire are capable of binding with a wide variety of antigenic determinants because of six hypervariable loops on their fragment variable domains called complementary determining regions (CDRs).

Molecular coevolution is the process of reciprocal evolutionary change that occurs between pairs of amino acids as they interact with one another. This property is observed when a mutation in one of two interacting amino acids, without a compensating mutation in the other amino acid, disrupts protein structure and negatively affects the fitness of the protein. Residue pairs for which there is a strong selective pressure to maintain mutual compatibility are therefore expected to mutate together or not at all. Discovering the molecular coevolution of antibody sequences is of high interest in order for scientists and engineers to explore how the adaptive immune system forms a protective network of biologically relevant antibodies to protect ourselves from infection and disease.

In applying context-dependent molecular coevolutionary analysis on antibodies, the prevailing assumption has been that antibody CDR regions possess limited epistatic dependencies because of their inherently high evolvability. Furthermore, an accurate direct couplings analysis approach would require a large multiple sequence alignment with coverage over the CDRH3 region, a non-canonical subregion, due to the forces of V-D-J gene recombination and affinity maturation. Fetching this many relevant sequences to inform a direct couplings analysis model is generally not possible because of CDRH3's highly divergent sequence identity. A BLAST search of CDRH3 subregion over an adaptive immune repertoire will return at best a handful of non-redundant sequences, and less so if the antibody sequence of interest is not native to the subject of the repertoire. This is because the immune system possesses limited commonality from subject to subject.

### SUMMARY

In a first aspect, sequence data is received that specifies at least one sequence of interest. Thereafter, homologous sequence are collected based on the sequence data and are represented in a multiple sequence alignment using a novel search approach. Next, an epistatic model is computed by a first machine learning model that represents a coevolutionary landscape of the multiple sequence alignment. Later, a second machine learning model is used to iteratively generate statistical inferences based upon the epistatic model, to result in a candidate pool of sequences comprising variants of the sequence of interest. Data can then be provided which characterizes the candidate pool of sequences.

The providing of data can take various forms including one or more of: causing the data to be displayed in an electronic visual display, loading the data characterizing the variants into memory, or transmitting the data characterizing the variants to a remote computing system,

A least one sequence of interest can be a biological sequence derived from sequencing biological matter or by computational design. The biological sequence can be, for example, a protein such an antibody. In other variations, the biological sequence can be an infectious pathogen.

The epistatic model can be a direct couplings analysis (DCA).

The epistatic model can be an undirected graphical model which represents co-evolutionary relationships.

The first machine learning model can take various forms including a state-based undirected graphical model.

The second machine learning model can include a model trained using reinforcement learning to perform site-directed in silico mutagenesis.

The second machine learning model can employ reinforcement learning on, for example, the coevolutionary landscape of the first machine learning model. In some variations, the reinforcement learning can comprise a Bayesian multi-armed bandit algorithm applied using a Dirichlet process over the coevolutionary landscape of the first machine learning model.

The homologous sequences can include related biological sequences described by one or more of: a domain similarity score, CDR length and/or CDR structural geometry, matching V-D-J germline genes to the sequence of interest, or similar biophysical characterizations.

The candidate pool of sequences can include a listing of biological sequences on a scale of no less than 1000, and in some cases, no more than 10⁷ sequences.

Model parameters of the epistatic model can be learned by employing an entropy maximization method. The effects of the mutations can then be learned from the model so that variants of the sequence of interest are iteratively with machine learning to maximize an inferred likelihood of positive natural selection for the variant.

The variants can be sequences bearing at least one amino acid or nucleotide mutations away from the sequence of interest.

The generating can be iteratively performed n times, resulting in first to nth order variants. In one example, n is five which can, in some variations, result in over one million (1,000,000) variants are initially generated as part of the iteratively generating.

In an interrelated aspect, data is received that specifies an antibody sequence of interest. Variants of the antibody sequence of interest are iteratively generate by an evolutionary mutagenesis model using the antibody sequence of interest. One or more filtering algorithms (e.g., screening algorithms) are used to select a subset of the iteratively generated variants of the antibody sequence of interest based on a likelihood of evolution. Thereafter, the selected subset of the iteratively generated variants of the antibody sequence of interest are computationally screened, in silico to result in a candidate antibody pool.

The one or more filtering algorithms can calculate, for each variant sequence of interest, a frequency of different amino acids at various positions, relative to a presence of other amino acids.

In yet another interrelated aspect, data is received that characterizes a candidate pool of variants of sequences. Thereafter, sequence liabilities are calculated for each of the variants in the candidate pool. Variants from the candidate pool that possess more sequence liabilities than an antibody seed sequence are eliminated. Later, structural properties of all variants in the candidate pool are computationally characterizing using at least one machine learning model. Biophysical patches are then characterized using predicted structural properties for each of the variants in the candidate pool. A statistical divergence of a structure of each of the variants in the candidate pool is calculated relative to the structure of at least one sequence of interest. The variants in the candidate pool are computationally screened using the structural properties and biophysical patches of the variants to result in screened variants. Data can then be provided (e.g., displayed, stored, loaded into memory, transmitted to a remote computing system, etc.) which characterizes the screened variants.

The at least one machine learning model can include a deep learning residual neural network, and in some variations, additionally include a long-short term memory network.

Variants in the candidate pool are compared for similarity with and divergence from proteins possessing at least one a desirable or undesirable biophysical characteristics. The biophysical patches on the variants in the candidate pool are compared with a target protein epitope for interaction likelihood. The computational screening can be based on both such comparisons.

The statistical divergence of the structure can be calculated based upon comparing distributions describing a geometry of a carbon backbone of the at least one sequence of interest.

The computational screening can include grouping variants into candidate clusters, based on at least genetic diversity, biophysical diversity, interaction likelihood, or structural diversity, computationally modeling biomolecular interactions between variants in the candidate clusters and antigens of interest, downsampling the variants in the candidate pool based on the computational modeling of the biomolecular interaction, and/or yielding a subset of the variants that existed in the candidate pool which comprise the screened variants.

The structural properties can include secondary, tertiary, and quaternary features.

The structural properties can include surface exposure and distance-relations of amino acids.

The candidate pool of variants of sequences can be generated by: receiving sequence data specifying at least one sequence of interest, collecting, based on the sequence data, homologous sequences and representing them in a multiple sequence alignment using a novel search approach, computing, by a first machine learning model, an epistatic model representing a coevolutionary landscape of the multiple sequence alignment, and iteratively generating, by a second machine learning model, statistical inferences based upon the epistatic model, to result in a candidate pool of variants of sequences comprising variants of the sequence of interest.

The providing data can include one or more of: causing at least a portion of the data to be displayed in an electronic visual display, storing at least a portion of the data in physical persistence, loading at least a portion of the data in memory, or transmitting at least a portion of the data to a remote computing system.

The computational screening can utilize characteristics including one or more of: hydrophobicity, polarity, charge, solubility, amino acid composition, isoelectric point, or disorder/entropy.

Non-transitory computer program products (i.e., physically embodied computer program products) are also described that store instructions, which when executed by one or more data processors of one or more computing systems, cause at least one data processor to perform operations herein. Similarly, computer systems are also described that may include one or more data processors and memory coupled to the one or more data processors. The memory may temporarily or permanently store instructions that cause at least one processor to perform one or more of the operations described herein. In addition, methods can be implemented by one or more data processors either within a single computing system or distributed among two or more computing systems. Such computing systems can be connected and can exchange data and/or commands or other instructions or the like via one or more connections, including but not limited to a connection over a network (e.g., the Internet, a wireless wide area network, a local area network, a wide area network, a wired network, or the like), via a direct connection between one or more of the multiple computing systems, etc.

The subject matter described herein provides many technical advantages. For example, the current subject matter provides advance techniques for the in silico discovery of antibodies and antibody-like biomolecules with therapeutic potential. In particular, the current subject matter is advantageous in that it provides a machine-learning approach to develop a representation of the molecular coevolutionary landscape of antibody CDRs which, in turn, can be used to generate a search space of evolutionarily-likely variant antibodies.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1 is a process flow diagram illustrating a first phase of a computational pipeline that is responsible for generating a pool of evolutionarily likely antibody variants;

FIG. 2 is a process flow diagram illustrating a second phase of the computational pipeline that is responsible for down-sampling the pool of antibody variants to a panel of antibodies having therapeutic potential;

FIG. 3 is a heat map representing the effects of first order mutations within the molecular coevolutionary landscape of an antibody's CDRH3 sequence;

FIG. 4 is a process flow diagram illustrating the computational directed evolution of protein sequences;

FIG. 5 is an image of predicted CDR loop structures of an example antibody heavy chain. Magenta loops are the result of the ResNet's predicted geometry for an antibody. Yellow loops are the ground truth of the original X-ray Crystallography for the same antibody. Cyan loops are the ResNet's predicted geometry for a variant sequence bearing a two mutations in CDRH3; and

FIG. 6 is a diagram illustrating aspects of a computing device for implementing the current subject matter.

### DETAILED DESCRIPTION

The current subject matter is directed to computationally deriving the patterns of evolution of biological sequences that yield a candidate panel targeting a protein or biological molecule of interest. The current subject matter provides a set of machine learning algorithms (a ‘pipeline’) which can be seeded with either: (a) a novel sequence designed in silico to interact with the target protein of interest; or (b) a known ligand to the target of interest. The machine learning pipeline comprises several machine learning methods that (a) generate relevant search space of variant sequences to the seed sequence; (b) infer characteristics and interaction potential; and (c) down-sample the search space to yield a therapeutic candidate pool.

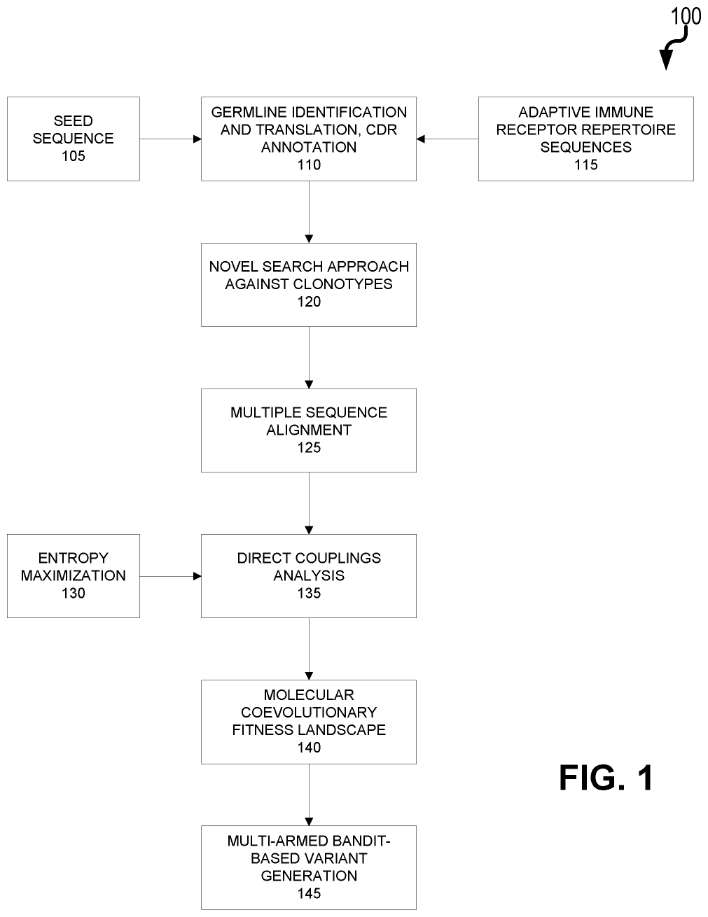

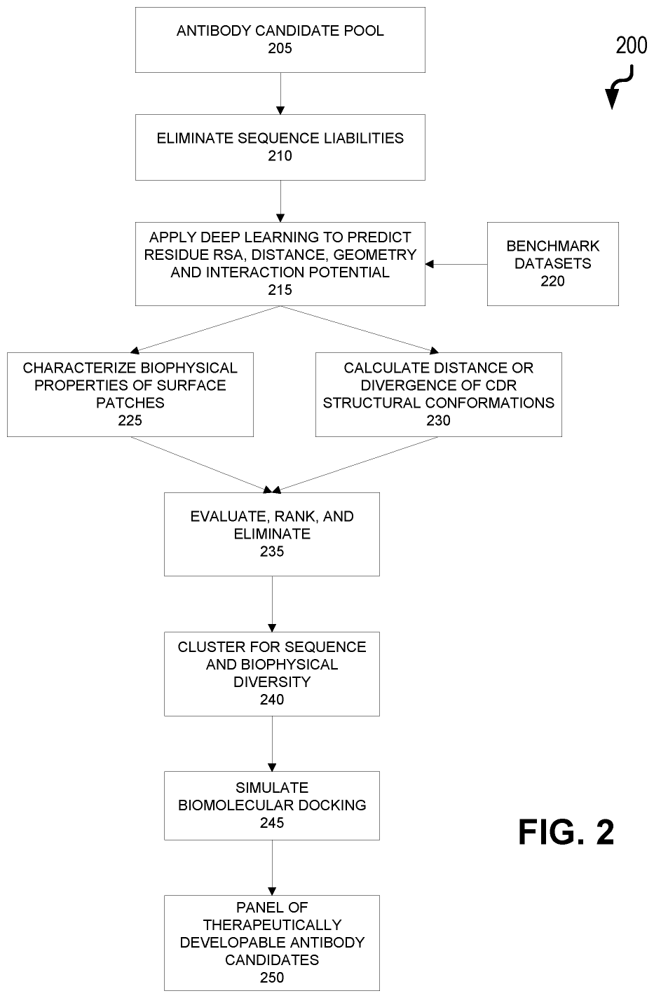

The current subject matter encompasses the discovery that: (ii) molecular coevolutionary information of antibody sequences, specifically at the key site of the CDRH3, can be extracted from an adaptive immune receptor repertoire in order to generate a pool of variant sequences (see FIG. 1), and (ii) the candidate pool can be directed through a series of computational modules in a pipeline to yield a therapeutically relevant antibody candidate panel (see FIG. 2).

With reference to diagram 100 of FIG. 1, at 105, the current subject matter utilizes a seed antibody sequence. The current subject matter can be used with DNA, RNA, amino acid sequences as well as antibody sequences (with antibody sequences being described for illustrative purposes). The seed antibody sequence may contain the fragment variable domain of an antibody or antibody-like biomolecule that includes one heavy and one light chain of lengths ranging 105 through 140 amino acids (or the DNA sequence representing the amino acids). The seed antibody sequence can take various forms, such as an single chain variable fragment (scFv), VHH, Fab-fragment, mAb, bi-specific, and may be of any isotype (e.g., IgG, IgM, IgA, or IgE). The seed antibody may originate from various sources including as a known binding antibody to a desired target discovered in the laboratory, an antibody identified from an adaptive immune receptor repertoire, and/or an antibody sequence computationally designed ab initio against a target of interest.

A multiple sequence alignment 125 can be constructed in order for the machine to generate a pool of variant sequences. The multiple sequence alignment 125 is prerequisite because molecular coevolutionary information of the seed antibody sequence can only be extracted by the machine learning models with a series of sequences possessing some degree of homology to the seed sequence, and specifically over the antibody's CDRH3 region.

Ordinarily, a phylogenetic multiple sequences alignment must be constructed in order to detect evolutionary covariation and to minimize statistical noise. An alignment with depth of at least several hundred sequences while still retaining alignment specificity (breadth of sequence coverage) over the domain of interest can be necessary. However, in the case of antibodies, conventional approaches using search tools (e.g., BLAST, HMMER3) fail to provide sufficient alignment coverage over the most important domain, the CDR3 of the antibody sequence heavy and light chains. Discovering the evolutionary constraints that exist within the CDR3 domain informs a machine learning model's ability to generate evolutionarily likely and biologically relevant CDR3 variants over this crucial domain and with insufficient alignment on this subregion, the evolutionary covariation learned by a machine learning model is generally statistical noise.

The following details a workflow that supports a novel approach to search 120 and construct a multiple sequence alignment 125 of biologically relevant antibodies to an antibody seed sequence 105.

A bulk sequenced adaptive immune repertoire (AIRR) 115 as provided herein can serve as an appropriate search database. The database can be further prepared by use of immunogenomic annotation tools (e.g., igBLAST, IMGT V-Quest, ANARCI). Germline genes for the antibody heavy chain can be identified, at 110, in the database and all antibody sequences can be numbered using the Kabat or Chothia annotation methods. Clonotypes within the database can then be identified. As defined herein, clonotypes are sequences possessing the same V, D, J genes and exactly the same CDRH3 sequence.

With clonotypes identified, a novel search approach can be executed, at 120, which does not rely exclusively upon sequence similarity search tools (e.g., HMMR3, BLAST), but instead employs both immunogenomic and biomolecular fingerprinting as measures of homology. The biomolecular fingerprinting include: (i) biophysical characteristics (e.g., hydrophobicity and charge), and (ii) structural characteristics (e.g., geometry, residue distances).

The novel search approach can include one or more of the following operations to find sequences with CDR3 subregions bearing immunogenomic and biomolecular homology.

(1) The database's clonotypes are compared to the clonotype of the seed sequence. At least several hundred thousand unique clonotypes of the same V, D, and J gene constructs can be identified. Synthetic expansion of the dataset can bolster the results of this search as previously described.

(2) Gather any clonotypes in this population which bear >30% CDR3 sequence identity to the seed sequence. This step is performed after (1) and not prior to (1) because CDR3's bearing high sequence identity but divergent germline V, D, and J genes obscure the DCA model's specificity for evolutionary covariation specific to the focus sequence of interest's CDR3.

(3) Apply biomolecular fingerprinting to the clonotypes in (1). The machine learning and deep learning based approaches to predicting biomolecular fingerprints is further detailed later on in this description. Sequences can be added to the alignment which bear high homology to the seed sequence of interest on the basis of their biomolecular fingerprint.

An example of one such approach follows:
(a) identify a subpopulation of clonotypes with CDR3 carbon backbone distance of less than or equal to 4 angstroms in length from the focus sequence's carbon backbone.
(b) of the subpopulation identified in the prior operation, compare the clonotype's patches of surface hydrophobicity and charge. Each of these patches can be described as a single, statistic measure.
(c) encode the sequence of the clonotypes by amino acid subgroup (i.e., aliphatic=A, polar=P, small=S, etc.) and calculate a sequence similarity to the focus sequence of interest encoded in the same manner.
(d) apply a deep learning network (e.g., residual neural network, etc.), to predict the inter-residue distances and angles (e.g., dihederal, omega, theta) of the CDR3 loops. One such deep learning network is described further in connection with FIG. 2. This can be used to compare the structural homology to the focus sequence of interest. The distance of angles or statistical divergence can be used a method of gathering structurally relevant clonotypes.

All of the above operations can form an arrangement in which biomolecular fingerprints can be formulated over a subpopulation of immunogenomic-relevant antibody sequences that have been naturally observed an AIRR, and/or synthetically derived from one.

To select clonotypes that bear high homology to the focus sequence of interest, sequence selection can be applied using a data science approach by mapping the biomolecular fingerprints into R-dimensional space such that each dimension describes a feature of the biomolecular fingerprint of a sequence. Similar work can be performed for structural predictions. A distance metric (e.g., Euclidean, Mahalanobis, etc.) can then be applied to these data points to determine those which sit closest to the focus sequence of interest. However, if the mapping is too highly dimensional, it is equally useful to manually set thresholds for inclusion and perform univariate analysis.

The multiple sequence alignment 125, should be of a depth of at least several hundred if not several thousand sequences, all with coverage over the CDR3. Whereas in the preparatory work entailed in 110 employed an immunogenomic annotation such as Chothia or Kabat numbering, a structure based antibody annotation can be applied on the multiple sequence alignment 125 (e.g., Aho scheme).

The above concludes the novel search approach 120 to gather a relevant multiple sequence alignment 125.

The multiple sequence alignment 125 can be consumed, at 135, by a statistical model that quantifies patterns of molecular coevolution from the multiple sequence alignment. Molecular coevolution can be mapped by various techniques including direct coupling analysis (DCA) statistical method. The objective of constructing a DCA based statistical method is to quantify the strength of the direct relationship between amino acid pairings in a biological sequence. These one-to-one amino acid pairings are referred to as couplings.

When fitted to a multiple sequence alignment of sequences of length N, the DCA model 135 defines a probability for all possible sequences of the same length. A sequence can be defined by a=(α₁, α₂ . . . , α_{N}) with the αᵢ being one of the 20 common amino acids, (and 21 if including a gap symbol). The probability of observing a sequence within a DCA model is then defined as (1):

$$P ( a \vert J , h ) = \frac{1}{z} exp ( {\sum }_{i = 1}^{N - 1} {\sum }_{j = i + 1}^{N} {J}_{ij} ( {a}_{i} , {a}_{j} ) + {\sum }_{i = 1}^{N} {h}_{i} ( {a}_{i} ) ) \qquad ( 1 )$$

where:

J, h are sets of real numbers representing the parameters of the model Z is a normalization constant to ensure that the sum of marginal probabilities is equal to 1.

The parameters hᵢ(αᵢ) depend on one position i and the symbol αᵢ at this position. This is the position-specific propensity to represent a particular amino acid. The parameters Jᵢⱼ(αᵢ, αⱼ) depend on the positions i, j and their respective amino acids αᵢ, αⱼ, at those positions.

The covariance matrix, Jᵢⱼ, can be generated by determining (e.g., counting) how often a particular pair of amino acids occurs in a particular pair of positions in any one sequence and summing over all sequences in the multiple sequence alignment.

If the multiple sequence alignment comprise evolutionarily related sequences, these direct quantifiable relationships represent evolutionary pressure for couplings to maintain mutual compatibility in the biomolecular structure of the sequence. Large values of couplings in a DCA model 135 indicates high evolutionary conservation between the two positions in the protein and indicates the property of molecular coevolution.

Ordinarily, the statistical parameters J, h in equation (1) could be inferred using maximum likelihood estimation, however the normalization constant Z needs to be calculated over sequence length N and 21 possible symbols, a sum of 21^{N} terms (e.g, 30 amino acid length sequences translates to 21³⁰ terms).

A well fitted DCA model must explain the observed data. There are a variety of alternative methods by which the DCA model 135 above can be fitted to the data in lieu of maximum likelihood estimation. The most appropriate method by which the DCA model, at 135, is fitted to the observed multiple sequence alignment data is by following the entropy maximization principle. Using this principle, it follows that the probability distribution which best represents the observable sequences in the multiple sequence alignment is the one with the largest Shannon entropy. The entropy maximization approach builds a probability model for an entire sequence by not assuming any additional information about non-observed sequences in the observed multiple sequence alignment 125. An entropy maximization approach finds the most uniform probability distribution that matches the observed sequences. The observed sequences of the multiple sequence alignment therefore serve as constraints for the parametrized DCA model.

The following details how coevolving pairs of residues are identified using maximum entropy 130 to infer the parameters of the above equation (1). Once a DCA model has been fit using entropy maximization, the quantifiable values which represent direct relationships of the couplings will be observed by the inferred parameters J, h.

A multiple sequence alignment 125 can be represented as an M×L matrix Aᵢ^{m} in which each row corresponds to one of m=1, . . . , M sequences and each column to one of i=1, . . . , L sequence positions. Each matrix element Aᵢ^{m} can be either one of the 20 standard amino acids or the gap character, and thus have q=21 different values.

Then, the frequency of an amino acid A in column i of the alignment is given by:

$${f}_{i} ( A ) ≡ \frac{1}{M} {\sum }_{m = 1}^{M} δ ( {A}_{i}^{m} , A ) \qquad ( 2 )$$

The frequencies of amino acid observations are obtained using the Kronecker-delta δ(a, b), which equals 1 if a=b, 0 else.

The frequency of an observed pair of amino acids A and B in columns i and j, respectively can be similarly defined as:

fᵢⱼ(A,B)=1/MΣₘ₌₁^{M}δ(Aᵢ^{m},A)δ(Aⱼ^{m},B) (3)

When the product of the individual frequency distributions fᵢ(A)fⱼ(B) are approximately equal to the joint frequency distribution fᵢⱼ(A, B), there is little if any correlation, while a deviation indicates a correlation between the two columns in the multiple sequence alignment.

The frequency counts of equations (2) and (3) can be redefined as:

$${f}_{ij} ( A , B ) ≡ \frac{1}{λ + {M}_{eff}} ( \frac{λ}{{q}^{2}} {{\sum }^{M}}_{m = 1} \frac{1}{{k}_{m}} δ ( {A}_{i}^{m} , A ) δ ( {A}_{j}^{m} , B ) )$$

where:
M_{eff} is the effective number of sequences in the alignment after weighting,
λ is the pseudocount term to avoid non-zero entries in the correlation matrix,
kₘ is a weight assigned to each sequence m, downweighting m for the number of identically aligned residues above a chosen threshold.

The parameters hᵢ(αᵢ) depend on one position i and the symbol αᵢ at this position. This is the position-specific propensity to represent a particular amino acid. The parameters Jᵢⱼ(αᵢ, αⱼ) depend on the positions i, j and their respective amino acids αᵢ, αⱼ, at those positions.

The entropy maximization 130 can take various forms including a pseudo-likelihood maximization. As part of such entropy maximization, an undirected graphical model of the coevolutionary landscape can be created which is described in further detail below and which is designed for the purpose of biological sequences. A fast Limited-memory Broyden-Fletcher-Goldfarb-Shanno algorithm (L-BFGS) maximum a posteriori estimation of the parameters using gradient descent can be performed. Further, l2 regularization can be performed to reduce high false positive rates due to indirect correlations, a problem inherent when describing correlation with measures like mutual information.

For the purpose of learning evolutionary constraints of homologous antibody sequences, and specifically with the intent of learning the search space of CDR3, it is recommended to set 0 weight on penalty parameter kₘ and to use all available sequences for M_{eff}. This is because the number of observed CDR3 bearing homology to the focus sequence are limited and the highly divergent sequence identity requires all observed sequences in order for the model to fully explain the data.

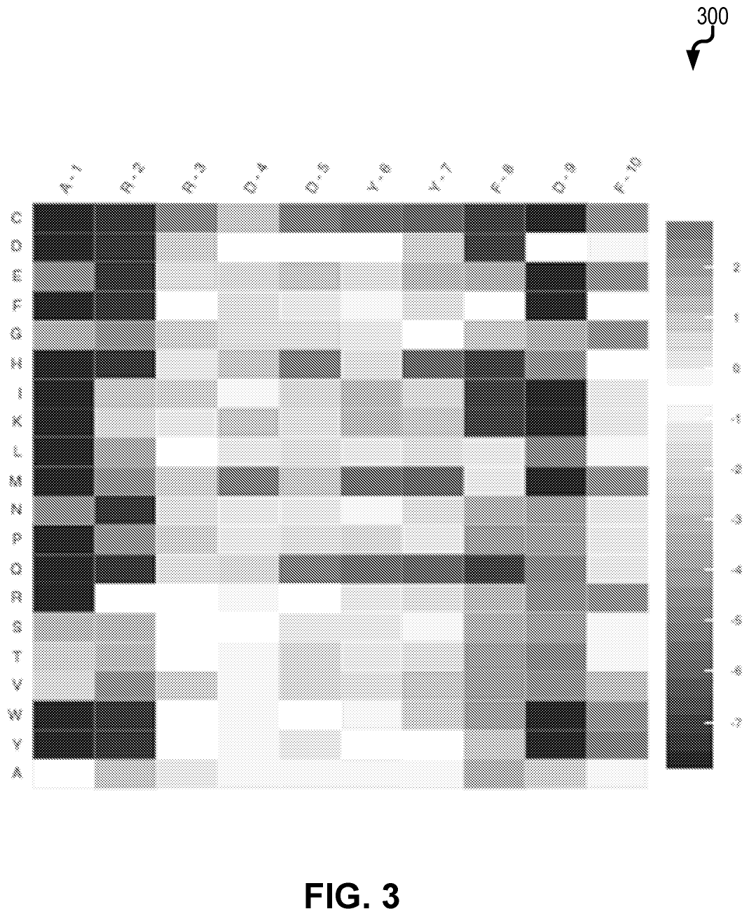

Once the parameters have been inferred by maximum entropy method 130, the effects of mutations as illustrated in the diagram 300 of FIG. 3 is represented statistically by the evolutionary couplings term (the covariance matrix) Jᵢⱼ(αᵢ, α₁) in formula (1) above. The programmatic application of equation (1) will calculate an evolutionary likelihood score for a series of residue changes i and j by measuring the differences in entropy between a learned distribution and the expected distribution under statistical independence, thereby providing a single, point estimate of the evolutionary likelihood for a set of mutations.

The coevolutionary landscape can be represented as a fully connected undirected graphical model G=(V, E) where the vertices V are the positions and the edges E are the evolutionary couplings. Such models can be based on Markov random fields. In one example, the edges between any two vertices are weighted by the pairing's evolutionary coupling score as identified by the parameters Jᵢⱼ(αᵢ, αⱼ), the value of the vertices is described by the term hᵢ(αᵢ). This model can be seen as a generalization of the Ising or Potts models. In the case of an Ising model, the spins will not only taking two values, but any value from a given finite alphabet. In fact, when the size of the alphabet is 2, the model reduces to the Ising model.

The conditional distribution of a set of mutations to the sequence of interest can be exactly inferred by considering the set of positions the mutations will be applied, V′={v₁, . . . , vᵢ}, given values to the non-mutated, positions W′={w₁, . . . , wⱼ} in the Markov random field by summing over all possible amino acid assignments aᵢ∉V′, W′. However, because exact inference of the search space is a #P-complete problem, as discussed earlier, a machine learning algorithm can be employed to explore and exploit the Markov random field at 145 to fetch the best sets of possible mutations.

The molecular coevolutionary fitness landscape, at 140, can be utilized, at 145, to generate variants optimized for high evolutionary likelihood. To do so, the effects of mutations must be predicted by sampling a set of mutations from the landscape. Consider the net evolutionary likelihood as an energy score, or a Hamiltonian in statistical physics. A Hamiltonian is an operator H which corresponds to the total energy of a system. The effect of any mutation could raise H (the net evolutionary likelihood score) of a sequence or decrease H when compared to the original wildtype sequence which has an H of zero. It is also possible that a mutation taken independently may have a negative effect on H, but in combination with other mutations in a set, results in a significantly higher evolutionary likelihood.

The problem of discovering the optimize sets of mutations that provide the most positive effect of evolutionary likelihood H can be framed as a Bayesian multi-armed bandit machine learning problem 145. Multi-armed bandit machine learning problems are classed under classic reinforcement learning algorithms. Consider the machine as the agent, tasked with the goal of finding the highest evolutionary likely set of mutations within the coevolutionary space. The agent has many competing or alternative choices it may pick to maximize its expected gain and the creation of one set of mutations comprises of a round. In a round, the agent will pick one mutation and append it to a set.

There is a limited set of independent losses to the evolutionary likelihood H score, that the agent will be allowed to take in choosing mutations. This serves to keep the agent from performing a greedy search of the space, whereby the agent chooses only mutations that have an immediate local improvement on the evolutionary likelihood. In this regard, the loss of evolutionary likelihood can be regarded as a limited resource, where at every round, the agent is allowed to suffer three consecutive losses before the round terminates.

In the problem, each mutation provides a random gain or loss from a probability distribution specific to that mutation. The objective of the agent is to maximize the sum of rewards earned through constructing a series of mutations. The crucial tradeoff the agent faces at each trial is between “exploitation” of the mutations that has the highest expected reward and “exploration” to get more information about the expected reward of the other mutations.

The multi-armed bandit can be seen as a set of real distributions B={R₁, . . . , R_{K}}, each distribution being associated with the rewards delivered by one of the K∈N⁺ mutations. Let μ₁, . . . , μ_{K} be the mean values associated with these reward distributions. The agent iteratively samples one mutation and observes the associated reward. The objective is to maximize the sum of the collected rewards. The horizon H is the number of rounds that remain to be played. The bandit problem is formally equivalent to a one-state Markov decision process. The loss p after T samples is defined as the expected difference between the reward sum associated with an optimal strategy and the sum of the collected rewards:

$$ρ = T {μ}^{*} - {\sum }_{t = 1}^{T} {\hat{r}}_{t} ,$$

where μ* is the maximal reward mean μ*= [symbol] {μₖ},
and \hat{r}ₜ is the reward in round T.

Dirichlet distributions can be used as prior distributions in Bayesian statistics. The infinite-dimensional generalization of the Dirichlet distribution is the Dirichlet process. A Dirichlet process is a probability distribution whose range is itself a set of probability distributions. It is often used in Bayesian inference to describe the prior knowledge about the distribution of random variables how likely it is that the random variables are distributed according to one or another particular distribution. The Dirichlet process is specified by a base distribution H and a positive real number a called the concentration parameter (also known as scaling parameter). The base distribution is the expected value of the process, i.e., the Dirichlet process draws distributions “around” the base distribution the way a normal distribution draws real numbers around its mean. The scaling parameter specifies how strong this discretization is: in the limit of α→0, the realizations are all concentrated at a single value, while in the limit of α→∞ the realizations become continuous. Between the two extremes the realizations are discrete distributions with less and less concentration as a increases.

Given: (1) A measurable set S, comprising of randomly chosen amino acid mutations, (2) a base probability distribution H which upon instantiation is a uniform prior, and (3) a positive real number a, the Dirichlet process DP(H, α) is a stochastic process whose sample path (or realization), drawn from the process by an infinite sequence of random variates (i.e., mutations), is a probability distribution over S, such that the following holds. For any measurable finite partition of S, denoted {Bᵢ}ᵢ₊₁ⁿ. In this case, the measurable partitions of S are the delta Hamiltonian energy score representing the evolutionary likelihood for each mutation applied to the antibody sequence.

$$if X \sim DP ( H , α )$$

$$then ( X ( {B}_{1} ) , … , X ( {B}_{n} ) ) \sim Dir ( α H ( {B}_{1} ) , … , α H ( {B}_{n} ) )$$

where Dir denotes the Dirichlet distribution and the notation X˜D means that the random variable X has the distribution D.

With reference to diagram 200 of FIG. 2, upon the conclusion of running the multiarmed bandit algorithm, an antibody candidate pool 205 is yielded (it will be appreciated that the teachings provided herein can be used with antibody candidate pools identified using other methods). The antibody candidate pool 205 comprises of sequences at a scale of 10² through 10¹⁰, depending on how long the machine agent was allowed to explore and exploit the mutation space.

Subsequently, the pool of antibodies sequences are immediately screened for common sequence liabilities (e.g., free cysteines, deamidation, isomerization, oxidation, etc.) to effectively eliminate sequence liabilities 210. These operations can use a regex based search. Sequences possessing more sequence liabilities than the original focus seed sequence are immediate thrown out of the pool.

To predict residue relative solvent accessibility (RSA), distance, geometry and interaction potential of a single antibody would conventionally require many compute hours of a macromolecular homology modeling using software such as Rosetta or HADDOCK. This process is unscalable in characterizing the antibody candidate pools.

In place of this process, deep learning models (i.e., residual neural networks, long short-term memory networks, BERT transformers) can be used to provide sufficiently accurate predictions of protein structure. Both AlphaFold and RaptorX have shown that inter-residue distances can be accurately learned from sequence and coevolutionary features at the CASP14 (Critical Assessment of Techniques for Protein Structure Prediction). Both approaches used deep residual network architectures with dilated convolutions to predict inter-residue distances, which provides a more complete structural description than contacts alone. Once trained and validated, these models have the benefit of being rapidly scalable, producing predictions about a protein sequence in seconds of computation time.

The sequences within the candidate pool that remain after sequence liability elimination 210, at 215, are then characterized using several deep learning approaches. The same characterization approach is taken to benchmark datasets 220, which to date comprise of therapeutic antibodies in clinical trials or FDA approved (serves as a positive control), AIRR antibody repertoires (serves as a positive control), and disprot disordered proteins which serve as a negative control.

Thereafter, at 225, biophysical properties of surface patches can be characterized. Ordinarily characterization of proteins can be performed in bioinformatics by calculating a moving average across the values of a linear sequence. The amino acids can be translated to numerical values according to widely agreed upon scales/charts of hydrophobicity or charge (e.g. Kyte & Doolittle hydrophobicity index, etc.). This approach often falls inaccurate at representing the actual biophysics of proteins because linear sequences fold into secondary, tertiary, and quaternary level structures.

Networks of surface exposed, neighboring amino acids which share the same biophysical profile (i.e., hydrophobic/philic, positive/negative charge) can be developed with structural information taken into account. Such networks can be described as surface exposed patches, which provide a unique biomolecular fingerprint to describe a protein or in the case of this computational pipeline. Deep learning as provided herein (e.g., neural networks, etc.) can be used to rapidly produce scalable patch based characterizations over on very large candidate pools. By applying deep learning 210 to predict RSA and distance, ˜8,000 sequence are accomplished can be accomplished within a short amount of time as compared to conventional techniques.

Distance or divergence of CDR structural conformations can, at 230, be calculated. A deep learning residual neural network has proven capable of predicting antibody CDR geometry. The geometric predictions comprise of the omega, theta, and phi angles, as well as inter-residue distances from Carbon-beta to Carbon-beta atoms throughout the entire sequence. Given these geometric characterizations over the millions of potential antibody variants in the candidate pool, one can create comparison by calculating distances or statistical divergence between the CDRs of the variant sequence(s) back to the original focus sequence's geometry. To accomplish this, it will be important to provide the machine indexing of the amino acid positions if the antibody variants are of a different length from the focus sequence. This ensures the same CDR regions are being compared.

Other data science operations can be performed by evaluating, ranking, and eliminating sequences at 235 on the basis of their biophysical patches and CDR geometry. In the case of the former, the fully characterized benchmark datasets at 220 can serve as controls by which the variant's biophysical profiles may be evaluated, ranked, and eliminated. In the case of the latter, distance/divergence calculations of the CDR geometry can be applied by reducing and/or eliminate variants possessing high structural divergence from the original focus sequence. By constraining the variant pool this way, it allows for variants possessing the closest conformation to the original seed sequence to persist through to the final panel, which should have a greater likelihood of interacting with the target protein. Various methods of evaluation, ranking, and elimination of sequences can be performed on the biophysical patches depending on the desired endpoints of the project.

The candidate pool after the evaluation ranking and elimination at 235 can, at 240, then be clustered. Sequence similarity based clustering methods such as tools provided in MMSEQS can be applied to the candidate pool. Other clustering approaches (e.g., K-means) can be applied to variants by using the single-point statistics that describe their biophysical patch profiles.

Thereafter, at 245, selections of antibody sequences can then be, optionally, homology modelled using macromolecular modeling and protein docking. This will provide further predictive accuracy in assessing and ranking the relevance of the sequences in relation to the intended target.

The result of the simulation is at, 250, a panel of therapeutically developable antibody candidates. With the combined operations of the evaluation, ranking and eliminating, the clustering for sequence and biophysical diversity, and the biomolecular docking simulations, final selections of the best variants are yielded into a panel of what models have predicted to be the most therapeutically developable, evolutionarily likely, and biologically relevant antibody variant sequences. The sequences can then be produced using protein engineering methods in a laboratory and then validated.

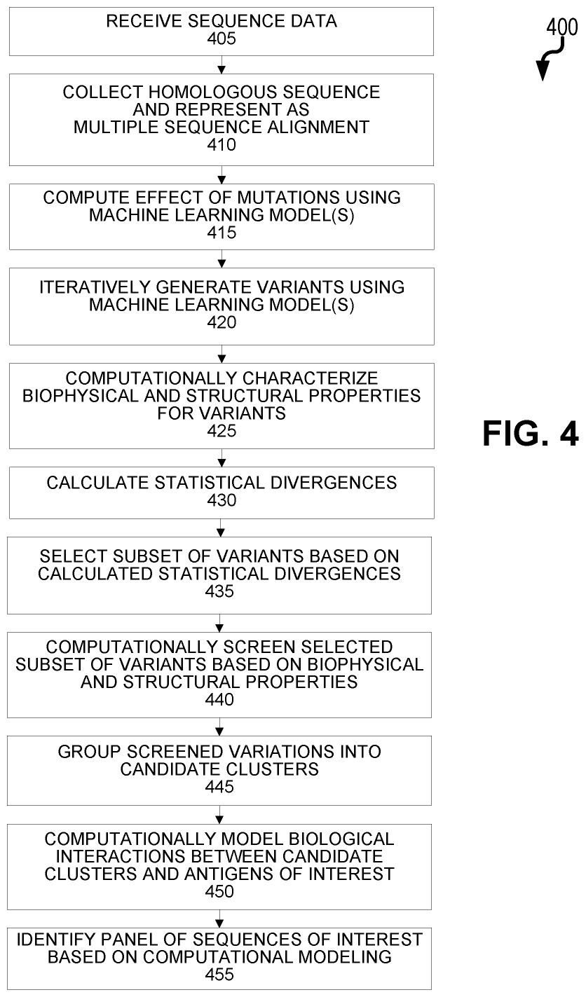

FIG. 4 is a diagram 400 illustrating the computational directed evolution of protein sequences, in which, at 405, sequence data is received that specifies at least one sequence of interest. Homologous sequences are collected, at 410, based on the sequence data and are represented in a multiple sequence alignment. Effects of mutations are computed, at 415, by instantiating a machine learning method that estimates parameters of an epistatic model representing homologous sequences in the multiple sequence alignment. Variants of the sequence of interested are then iteratively generating, at 420, using a machine learning model trained by applying reinforcement learning. Biophysical and structural properties can then be computationally characterized, at 425, for the variants. Using these computationally characterized structural properties, at 430, a statistical divergence of a structure of the variant relative to a structure of at least one sequence of interest can be calculated for at least one some of the variants. A subset of the variants is then selected, at 435, based on the calculated statistical divergences. Subsequently, at 440, the subset of the variant is computationally screened using both the characterized biophysical and structural properties of the selected subset of the variants, to result in a candidate pool of variants. These variants are later grouped, at 445, into candidate clusters, based on at least genetic diversity, biophysical diversity, or structural diversity. Biological interactions between variants in the candidate clusters and antigens of interest, are at 450, computationally modeled. Thereafter, at 455, data characterizing a panel of sequences of interest is compiled for laboratory development and experimentation from the candidate pool based on the computational modeling of the biological interactions.

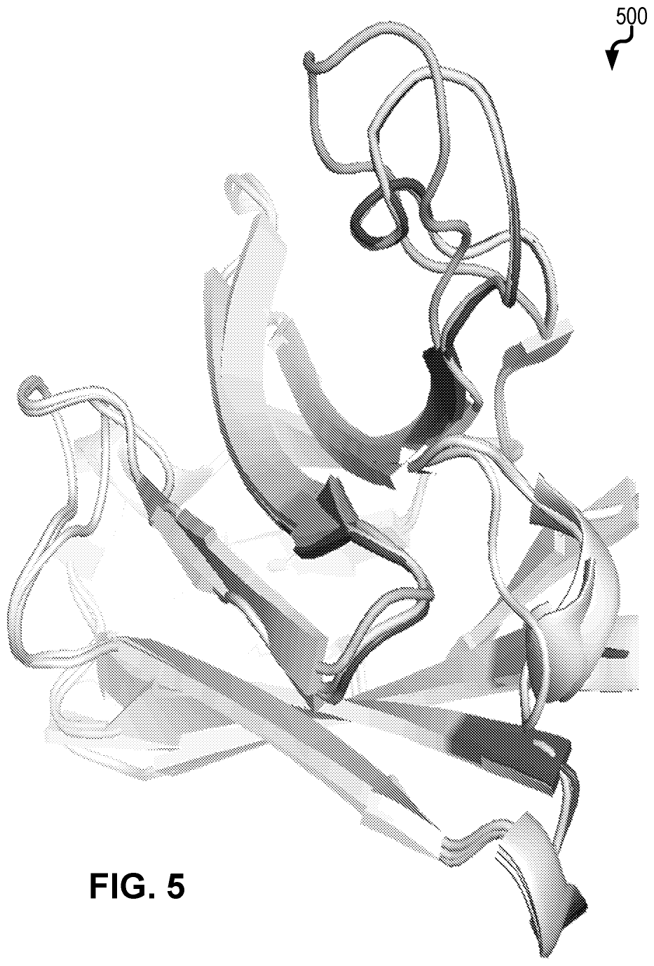

FIG. 5 is a diagram 500 illustrating predicted CDR loop structures of an example antibody heavy chain. Magenta loops are the result of the ResNet's predicted geometry for an antibody. Yellow loops are the ground truth of the original X-ray Crystallography for the same antibody. Cyan loops are the ResNet's predicted geometry for a variant sequence bearing a two mutations in CDRH3.

In one aspect, sequence data is received that specifies at least one sequence of interest. Thereafter, homologous sequence are collected based on the sequence data and are represented in a multiple sequence alignment using a novel search approach. Next, an epistatic model is computed by a first machine learning model that represents a coevolutionary landscape of the multiple sequence alignment. Later, a second machine learning model is used to iteratively generate statistical inferences based upon the epistatic model, to result in a candidate pool of sequences comprising variants of the sequence of interest. Data can then be provided (e.g., displayed, stored, loaded into memory, transmitted to a remote computing system, etc.) which characterizes the candidate pool of sequences.

In another aspect, data is received that specifies an antibody sequence of interest. Variants of the antibody sequence of interest are iteratively generate by an evolutionary mutagenesis model using the antibody sequence of interest. One or more filtering algorithms (e.g., screening algorithms) are used to select a subset of the iteratively generated variants of the antibody sequence of interest based on a likelihood of evolution. Thereafter, the selected subset of the iteratively generated variants of the antibody sequence of interest are computationally screened, in silico to result in a candidate antibody pool.

In another aspect, data is received that characterizes a candidate pool of variants of sequences. Thereafter, at sequence liabilities are calculated for each of the variants in the candidate pool. Variants from the candidate pool that possess more sequence liabilities than an antibody seed sequence are eliminated. Later, structural properties of all variants in the candidate pool are computationally characterizing using at least one machine learning model. Biophysical patches are then characterized using predicted structural properties for each of the variants in the candidate pool. A statistical divergence of a structure of each of the variants in the candidate pool is calculated relative to the structure of at least one sequence of interest. The variants in the candidate pool are computationally screened using the structural properties and biophysical patches of the variants to result in screened variants. Data can then be provided (e.g., displayed, stored, loaded into memory, transmitted to a remote computing system, etc.) which characterizes the screened variants.

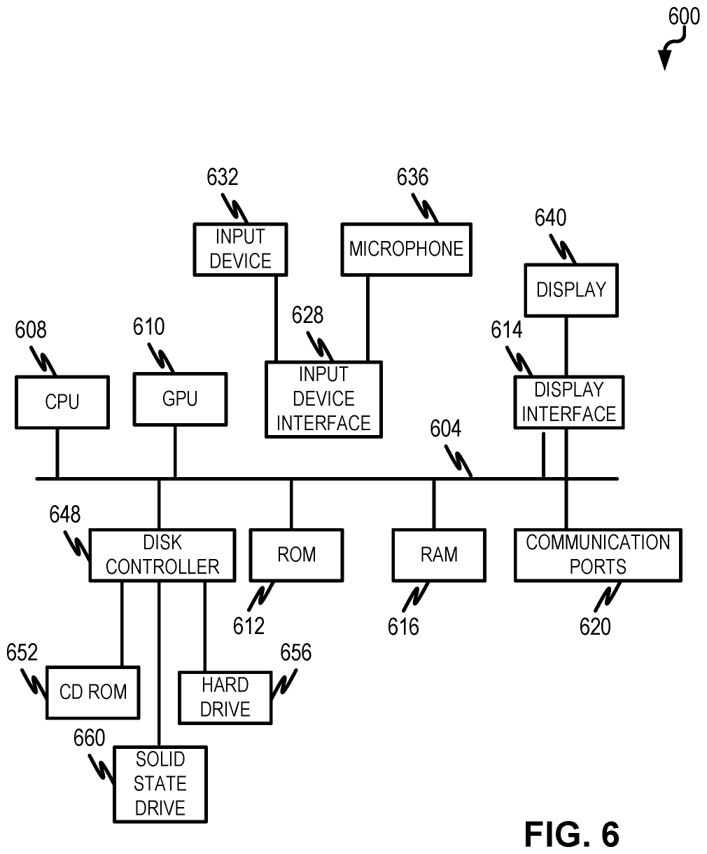

FIG. 6 is a diagram 600 illustrating a sample computing device architecture for implementing various aspects described herein. A bus 604 can serve as the information highway interconnecting the other illustrated components of the hardware. A processing system 608 labeled CPU (central processing unit) (e.g., one or more computer processors/data processors at a given computer or at multiple computers) and/or a GPU 610 (graphics processing unit) can perform calculations and logic operations required to execute a program. A non-transitory processor-readable storage medium, such as read only memory (ROM) 612 and random access memory (RAM) 616, can be in communication with the processing system 608 and can include one or more programming instructions for the operations specified here. Optionally, program instructions can be stored on a non-transitory computer-readable storage medium such as a magnetic disk, optical disk, recordable memory device, flash memory, or other physical storage medium.

In one example, a disk controller 648 can interface with one or more optional disk drives to the system bus 604. These disk drives can be external or internal floppy disk drives such as 660, external or internal CD-ROM, CD-R, CD-RW or DVD, or solid state drives such as 652, or external or internal hard drives 656. As indicated previously, these various disk drives 652, 656, 660 and disk controllers are optional devices. The system bus 604 can also include at least one communication port 620 to allow for communication with external devices either physically connected to the computing system or available externally through a wired or wireless network. In some cases, the at least one communication port 620 includes or otherwise comprises a network interface.

To provide for interaction with a user, the subject matter described herein can be implemented on a computing device having a display device 640 (e.g., a CRT (cathode ray tube) or LCD (liquid crystal display) monitor) for displaying information obtained from the bus 604 via a display interface 614 to the user and an input device 632 such as keyboard and/or a pointing device (e.g., a mouse or a trackball) and/or a touchscreen by which the user can provide input to the computer. Other kinds of input devices 632 can be used to provide for interaction with a user as well; for example, feedback provided to the user can be any form of sensory feedback (e.g., visual feedback, auditory feedback by way of a microphone 636, or tactile feedback); and input from the user can be received in any form, including acoustic, speech, or tactile input. The input device 632 and the microphone 636 can be coupled to and convey information via the bus 604 by way of an input device interface 628. Other computing devices, such as dedicated servers, can omit one or more of the display 640 and display interface 614, the input device 632, the microphone 636, and input device interface 628.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof: These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1. A computer-implemented method comprising:
receiving a candidate pool of variants of sequences;
calculating sequence liabilities for each of the variants in the candidate pool;
eliminating variants from the candidate pool that possess more sequence liabilities than an antibody seed sequence;
computationally characterizing, using at least one machine learning model and after the eliminating, structural properties of all variants in the candidate pool;
computationally characterizing biophysical patches using predicted structural properties for each of the variants in the candidate pool;
calculating a statistical divergence of a structure of each of the variants in the candidate pool relative to the structure of at least one sequence of interest;
computationally screening, using the structural properties and biophysical patches of the variants, the variants in the candidate pool to result in screened variants; and
providing data characterizing the screened variants.

2. The method of claim 1, wherein the at least one machine learning model comprises a deep learning residual neural network.

3. The method of claim 2, wherein the at least one machine learning model further comprises a long-short term memory network.

4. The method of claim 3 further comprising:
first comparing the variants in the candidate pool for similarity with and divergence from proteins possessing at least one a desirable or undesirable biophysical characteristics;
second comparing the biophysical patches on the variants in the candidate pool with a target protein epitope for interaction likelihood;
wherein the computational screening is based on the first comparing and the second comparing.

5. The method of claim 1, wherein the statistical divergence of the structure is calculated based upon comparing distributions describing a geometry of a carbon backbone of the at least one sequence of interest.

6. The method of claim 1, wherein the computational screening comprises:
grouping variants into candidate clusters, based on at least genetic diversity, biophysical diversity, interaction likelihood, or structural diversity.

7. The method of claim 6 further comprising:
computationally modeling biomolecular interactions between variants in the candidate clusters and antigens of interest.

8. The method of claim 7 further comprising:
downsampling the variants in the candidate pool based on the computational modeling of the biomolecular interaction.

9. The method of claim 8 further comprising:
yielding a subset of the variants that existed in the candidate pool which comprise the screened variants.

10. The method of claim 1, wherein the structural properties comprise secondary, tertiary, and quaternary features.

11. The method of claim 1, wherein the structural properties comprise surface exposure and distance-relations of amino acids.

12. The method of claim 1, wherein the candidate pool of variants of sequences are generated by:
receiving sequence data specifying at least one sequence of interest;
collecting, based on the sequence data, homologous sequences and representing them in a multiple sequence alignment using a novel search approach;
computing, by a first machine learning model, an epistatic model representing a coevolutionary landscape of the multiple sequence alignment;
iteratively generating, by a second machine learning model, statistical inferences based upon the epistatic model, to result in a candidate pool of variants of sequences comprising variants of the sequence of interest.

13. The method of claim 1, wherein the providing data comprises one or more of: causing at least a portion of the data to be displayed in an electronic visual display, storing at least a portion of the data in physical persistence, loading at least a portion of the data in memory, or transmitting at least a portion of the data to a remote computing system.

14. The method of claim 1, wherein the computational screening utilizes characteristics including one or more of: hydrophobicity, polarity, charge, solubility, amino acid composition, isoelectric point, or disorder/entropy.

15. A computer-implemented method comprising:
receiving a candidate pool of variants of sequences;
calculating sequence liabilities for each of the variants in the candidate pool;
eliminating variants from the candidate pool that possess more sequence liabilities than an antibody seed sequence;
computationally characterizing, using at an ensemble of machine learning models including a neural network and a long-short term memory network and after the eliminating, structural properties of all variants in the candidate pool;
describing biophysical patches using predicted structural properties for each of the variants in the candidate pool;
calculating a statistical divergence of a structure of each of the variants in the candidate pool relative to the structure of at least one sequence of interest;
computationally screening, using the structural properties and biophysical patches of the variants, the variants in the candidate pool to result in screened variants; and
providing data characterizing the screened variants.

16. The method of claim 15 further comprising:
first comparing the variants in the candidate pool for similarity with and divergence from proteins possessing at least one a desirable or undesirable biophysical characteristics;
second comparing the biophysical patches on the variants in the candidate pool with a target protein epitope for interaction likelihood;
wherein the computational screening is based on the first comparing and the second comparing.

17. The method of claim 16, wherein the statistical divergence of the structure is calculated based upon comparing distributions describing a geometry of a carbon backbone of the at least one sequence of interest.

18. The method of claim 17, wherein the computational screening comprises:
grouping variants into candidate clusters, based on at least genetic diversity, biophysical diversity, interaction likelihood, or structural diversity.

19. The method of claim 18 further comprising:
computationally modeling biomolecular interactions between variants in the candidate clusters and antigens of interest.

20. The method of claim 19 further comprising:
downsampling the variants in the candidate pool based on the computational modeling of the biomolecular interaction; and
yielding a subset of the variants that existed in the candidate pool which comprise the screened variants.

21-22. (canceled)
